(19) United States
(12) Patent Application Publication
(10) Pub. No.: US 2026/0216504 A1
(43) Pub. Date: Jul. 30, 2026

(54) FIBER SPECIFIC FOCUSED DIRECT CURRENT NEUROMODULATION

(71) Applicant: Case Western Reserve University, Cleveland, OH (US)

(72) Inventors: David Green, Cleveland, OH (US); Shane Bender, Cleveland, OH (US); Varun Thakkar, Cleveland, OH (US); Hope Zimmerman, Cleveland, OH (US); Mohamed Elzab, Cleveland, OH (US); Tina Vrabec, Cleveland, OH (US); Niloy Bhadra, Cleveland, OH (US)

(21) Appl. No.: 19/459,081

(22) Filed: Jan. 26, 2026

Related U.S. Application Data
(60) Provisional application No. 63/751,509, filed on Jan. 30, 2025.
(60) Provisional application No. 63/887,661, filed on Sep. 25, 2025.

(51) Int. Cl. A61N 1/20; A61N 1/05

(52) U.S. Cl. CPC A61N 1/205; A61N 1/0556

(57) ABSTRACT

One or more fibers in a nerve can be selectively stimulated and/or blocked with focused direct current (DC) neuromodulation based on application of a DC via at least one electrode coupled to a current generator. The current generator can generate at least one DC with an amplitude of less than 15 mA for a time and the at least one electrode can apply the DC to a nerve for the time. Application of the DC to the nerve for the time can create a spatially restricted DC field based on the DC and specific geometries and configurations of the at least one electrode. For instance, the spatially restricted DC field can at least partially block conduction in small-diameter nerve fibers of the nerve while leaving large-diameter, myelinated nerve fibers of the nerve substantially unblocked and able to conduct.

### CROSS-REFERENCE TO RELATED APPLICATIONS

This application claims the benefit of U.S. Provisional Application Ser. No. 63/751,509, filed 30 Jan. 2025, entitled “FIBER SPECIFIC FOCUSED DIRECT CURRENT NEUROMODULATION (FS-FDN)”, and also claims the benefit of U.S. Provisional Application Ser. No. 63/887,661, filed 25 Sep. 2025, entitled “FIBER SPECIFIC FOCUSED DIRECT CURRENT NEUROMODULATION”. The entirety of these provisional applications are incorporated by reference for all purposes.

### GOVERNMENT FUNDING

This invention was made with government support under NS116009 awarded by the National Institutes of Health. The government has certain rights in the invention.

### TECHNICAL FIELD

The present disclosure relates generally to electrical neuromodulation, and more specifically, to systems and methods for focused direct current (DC) neuromodulation to create a spatially-restricted DC field, changing the recruitment order of neural fibers.

### BACKGROUND

Extracellular electrical neuromodulation generally refers to applying a current to alter neural activity on or within extracellular space surrounding the nerve fiber. Neural activity can be altered to treat pain, autonomic conduction, spasticity, and the like. The alteration can be based at least partially on nerve fiber's activation threshold. The activation threshold generally refers to the minimum level of electrical stimulation required for a nerve fiber to produce a response. Generally, myelinated fibers have lower activation thresholds compared to unmyelinated fibers. Similarly, within similar fiber groups, large diameter fibers have lower activation threshold than smaller diameter fibers. So electrical neuromodulation affects large diameter and/or myelinated fibers (e.g., motor fibers, sensory fibers, and the like) before small diameter (unmyelinated or thinly myelinated) fibers (responsible for pain, autonomic conduction, spasticity, and the like). In other words, small diameter and/or unmyelinated fibers require higher amplitudes of current to cause a block and these higher amplitudes of current also block larger diameter and/or myelinated fibers. Accordingly, the electrical order of recruitment is opposite the physiological order of recruitment.

One of the primary challenges in electrically modulating small fibers for pain control, autonomic regulation, spasticity control, or the like, is getting around the electrical order of recruitment. Previous work to get around the electrical order of recruitment to achieve selective activation and/or block of small fibers has achieved only limited success and/or risks of permanent nerve damage. This previous work includes investigation into high frequency nerve block, anodic block, subthreshold depolarizing prepulses, and nerve cuff electrodes with very small inter-contact spacing to selectively activate one or more small diameter fibers. Current methods for electrical extracellular nerve block, using high frequency waveforms or direct current, continue to run into the problem that large-diameter, myelinated fibers have a lower block threshold compared to small-diameter and/or unmyelinated fibers.

### SUMMARY

The present disclosure describes a size selective block of one or more fibers within a nerve that overcomes the electrical order of recruitment (e.g., can block smaller diameter fibers and/or unmyelinated fibers before blocking myelinated fibers and/or large diameter fibers). The size selective block is created by focused direct current neuromodulation. Focused direct current neuromodulation can be performed with specific electrode designs and/or configurations to reverse the current amplitude to fiber type/diameter relationship such that Aδ and C nociceptive fibers can be blocked at lower current amplitudes than the large motor and/or Aα/β sensory fibers. Thus, the extracellular direct current neuromodulation can selectively block smaller and/or unmyelinated fibers (for pain control, autonomic regulation, spasticity control, or the like), while preserving conduction for tractile sensitivity and voluntary movement (in larger fibers and/or myelinated fibers).

In one aspect, the present disclosure includes a system for focused direct current neuromodulation. The system can include a current generator and at least one electrode coupled to the current generator and in electrical communication with at one nerve. The current generator can generate at least one direct current (DC) with an amplitude of less than 15 mA for a time. The at least one electrode can apply the DC to the nerve for the time. Application of the DC to the nerve for the time can create a spatially restricted DC field based on the DC and the at least one electrode. The spatially restricted DC field can at least partially block conduction in small-diameter nerve fibers of the at least one nerve while leaving large-diameter, myelinated nerve fibers of the at least one nerve able to continue conduction.

In another aspect, the present disclosure includes a method for focused direct current neuromodulation. The method can block conduction in small-diameter nerve fibers while leaving large-diameter, myelinated nerve fibers at least partially unblocked and able to conduct. The method includes generating, by a current generator, at least one direct current (DC) with an amplitude less than 15 mA for a time and applying, by at least one electrode coupled to the current generator, the DC to at least one nerve, including the small-diameter nerve fibers and the large-diameter, myelinated nerve fibers, for the time. Application of the DC to the nerve for the time creates a spatially restricted DC field that at least partially blocks conduction in the small-diameter nerve fibers of the nerve while leaving the large-diameter, myelinated nerve fibers of the at least one nerve able to conduct.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram of a system that can provide focused direct current neuromodulation to selectively block smaller and/or unmyelinated fibers (for pain control, autonomic regulation, spasticity control, or the like), while preserving conduction for tractile sensitivity and voluntary movement (in large diameter, myelinated fibers);

FIG. 2 includes illustrations of part of the system of FIG. 1 in use on a nerve including both afferent and efferent fibers;

FIG. 3 is an illustration showing the effects of a broad electric field and a focused electric field on conduction in myelinated and unmyelinated nerves;

FIG. 4 is an illustration of a configuration of the electrode of the system of FIG. 1;

FIG. 5 is an illustration of another configuration of the electrode of the system of FIG. 1;

FIGS. 6 and 7 are process flow diagrams illustrating methods related to focused direct current neuromodulation; and

FIGS. 8-10 are illustrations and graphical results of experiments for focused direct current neuromodulation.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “neuromodulation” refers to the alteration of conduction in one or more nerves using one or more therapy modalities to cause an effect. In some instances, the altered conduction caused by the neuromodulation can be reversible and/or adjustable. Although neuromodulation can be achieved via many modalities (e.g., electrical, light, heat, magnetic, pharmaceutical, etc.), the electrical modality is used herein. Accordingly, the term neuromodulation (when used herein) can be understood to refer to electrical neuromodulation—the application of an electrical signal (having a current and/or voltage) to achieve altered nerve conduction. Additionally, the neuromodulation described herein can be delivered extracellularly to the space surrounding the nerve rather than into an interior of the nerve.

As used herein, the term “current” refers to the flow of electric charge (e.g., electrons) through a conductor. Current can be direct current (DC) or alternating current (AC). Current (i) and voltage (V) are related by the equation V=iR, where R is resistance.

As used herein, the term “direct current” or “DC” refers to one directional flow of electric charge. The direct current can be described with respect to a DC waveform generated by a current generator. A DC electric field or stationary electric field is associated with the DC current and represents a constant electric field with no change in direction or magnitude over time.

As used herein, the term “alternating current” or “AC” refers to the flow of electric charge that reverses its direction and changes its magnitude periodically.

As used herein, the term “block”, or “DC block”, refers to the purposeful alteration of conduction via neuromodulation with a DC waveform configured to decrease (or down-regulate) conduction of at least one nerve fiber in at least one nerve.

As used herein, the term “nervous system” refers to a network of nerve cells and fibers that transmits impulses between parts of the body. The nervous system can include, but is not limited to, the somatic nervous system and the autonomic nervous system.

As used herein, the term “somatic nervous system” or “SNS” can refer to a part of the peripheral nervous system that is responsible for carrying sensory inputs to the brain and motor control commands to the muscles (e.g., skeletal muscles).

As used herein, the term “autonomic nervous system” or “ANS” can refer to a part of the peripheral nervous system that is responsible for control of involuntary bodily functions (e.g., that are not consciously directed) such as breathing, heartbeat, digestive processes, and the like.

As used herein, the term “nerve”, “nerve cell”, “neuron”, or the like, refers to a bundle of nerve fibers that send messages from parts of the body to the brain and/or vice versa in the form of electrical signals. For example, a nerve can include autonomic fibers (e.g., sympathetic fibers and/or parasympathetic fibers), motor fibers, sensory fibers, and/or a mixture of different types of fibers.

As used herein, the term “nerve fiber” or “fiber” refers to an axon, which is a long slender projection of neuron having a diameter that corresponds to conduction velocity. A nerve fiber conducts electrical impulses transmitting information in one or more directions throughout the body and is classified depending on the type of fiber (e.g., sympathetic, parasympathetic, sensory, motor, etc.), the diameter of the fiber, and/or if myelin coating is present. It should be understood that fibers can be of a “large” diameter or a “small” diameter and may be myelinated or unmyelinated/thinly myelinated.

Examples of “small-diameter nerve fibers” can include Ad nerve fibers (diameter 1-5 μm conduction velocity 3-30 m/s, thinly myelinated, associated with free nerve endings of tough and pressure, nociceptors of the neospinothalamic tract, cold thermoreceptors), B nerve fibers (diameter 1-5 μm, conduction velocity 3-15 m/s, myelinated, preganglionic fibers), and C nerve fibers (diameter 0.2-1.5 μm, conduction velocity 0.5-2.0 m/s, unmyelinated, associated with nociceptors of the paleospinothalmic tract and warmth receptors). Small-diameter nerve fibers can provide conduction of sensory information such as pain, spastic movements, autonomic regulatory information, or the like. Generally, small-diameter nerve fibers may be unmyelinated or thinly myelinated.

Examples of “large-diameter, myelinated nerve fibers” can include Aα (diameter 13-20 μm, conduction velocity 80-120 m/s, myelinated, associated with muscle spindle fibers and Golgi tendon organ), Aβ (diameter 6-12 μm, conduction velocity 33-75 m/s, myelinated, associated with all cutaneous mechanoreceptors), and Aγ (diameter 5-8 μm, conduction velocity 4-24 m/s, myelinated, associated with intrafusal muscle fibers). Large-diameter, myelinated nerve fibers can be motor or sensory fibers and can generally carry signals related to touch, vibration, proprioception, and muscle control.

As used herein, the term “node(s) of Ranvier” refers to one or more gaps in a myelin sheath surrounding myelinated nerve fiber where the axolemma is exposed to the extracellular space between the myelin sheaths (also called internodes). Neural conduction occurs between the nodes of Ranvier, increasing the speed of conduction compared to unmyelinated fibers. One of the primary components of a Node of Ranvier is sodium and potassium voltage-gated ion channels, which effect the conduction capabilities of a nerve. The size and the spacing of the internodes (and thus the number of nodes of Ranvier in a length of nerve fiber) vary with the fiber diameter in a curvilinear relationship that is optimized for maximal conduction velocity. The size of the nodes span from 1-2 μm whereas the internodes can be up to (and occasionally even greater than) 1.5 millimeters long, depending on the fiber type. At least 4 to 5 Nodes of Ranvier need to be blocked in a large-diameter, myelinated nerve to create a nerve block.

As used herein, the terms “patient” and “subject” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

Neuromodulation can be used to treat pain, spasticity, autonomic dysfunction, and the like. In order to treat pain, spasticity, autonomic dysfunction, and the like, traditional treatments often affect most or all fibers in a given nerve—small and large, and myelinated or unmyelinated—because current neuromodulation techniques operate according to a nerve fiber's activation threshold (the minimum level of electrical stimulation required for a nerve fiber to produce a response). Generally, according to the electrical order of recruitment, myelinated fibers have lower activation thresholds compared to unmyelinated fibers and large-diameter fibers have lower activation thresholds than small-diameter fibers. According to the electrical order of recruitment, large-diameter, myelinated fibers (e.g., motor fibers, sensory fibers, and the like) are affected before small diameter fibers (e.g., responsible for pain, autonomic conduction, spasticity, and the like), making it difficult to activate smaller fibers selectively. This electrical order of recruitment is opposite from the body's physiological order of recruitment (where, in order to provide smooth muscle contraction, the body recruits small motor fibers before large motor fibers).

In the clinical treatment of pain, spasticity, autonomic dysregulation, and the like it would be beneficial to be able to affect small-diameter fibers with electrical signals having a lower amplitude to stop and/or lessen the pain, spasticity, autonomic dysregulation, or the like, while maintaining sensation, volitional motor control, other physiological autonomic regulations and the like. One of the primary challenges in electrically modulating small fibers for pain control, autonomic regulation, spasticity control, or the like, is getting around the electrical order of recruitment. Previous work to achieve selective activation and/or block of small fibers has had only limited success. This previous work includes investigation into high frequency nerve block, anodic block, subthreshold depolarizing prepulses, and nerve cuff electrodes with very small inter-contact spacing to selectively activate small fibers and/or axons. However, current methods for extracellular nerve block, whether using high frequency waveforms or direct current, show similar relationships as in activation: myelinated axons and large diameter axons have lower block thresholds in similar axon groups.

The systems and methods described herein present specialized electrode designs and configurations that can form focused direct current neuromodulation to reverse the current amplitude to axon type/diameter relationship. The focused direct current neuromodulation creates a narrow electrical field compared to traditional neuromodulation. The narrow electrical field can be achieved in one of two ways: by a small diameter electrode placed directly on the nerve, or by shaping the DC field so that only a small part of the nerve is affected. By affecting only a small part of the nerve the electrical field only affects small-diameter nerve fibers and not large-diameter, myelinated nerve fibers. Thus, small-diameter nerve fibers, such as Aδ, B, and C fibers, can be blocked at lower current amplitudes than the large motor and sensory fibers, such as Aα and Aβ fibers; and selective block of only small diameter nerve fibers can be achieved while preserving conduction within the large-diameter, myelinated nerve fibers (e.g., large motor and sensory fibers).

### III. Systems

Focused direct current (DC) neuromodulation can create fiber size selective conduction blocks within a nerve by taking advantage of: varying conduction velocities across fiber types and sizes; varying internodal distances; and the differences between saltatory (myelinated) and continuous (unmyelinated) conduction. Focused direct current neuromodulation creates a depolarized region in nerve fibers that is narrower than the span of the number of nodes of Ranvier needed to block large-diameter, myelinated nerve fibers. However, this depolarized region is still sufficient to block unmyelinated small-diameter nerve fibers that have continuous conduction as well as slow conduction velocities. Therefore, a spatially restricted DC field with a lower amplitude than required by traditional neuromodulation therapies can prevent conduction in small-diameter nerve fibers (e.g., Aδ, B, and/or C) without affecting the minimum nodes to block conduction in large-diameter, myelinated nerve fibers (e.g., Aα and/or Aβ fibers).

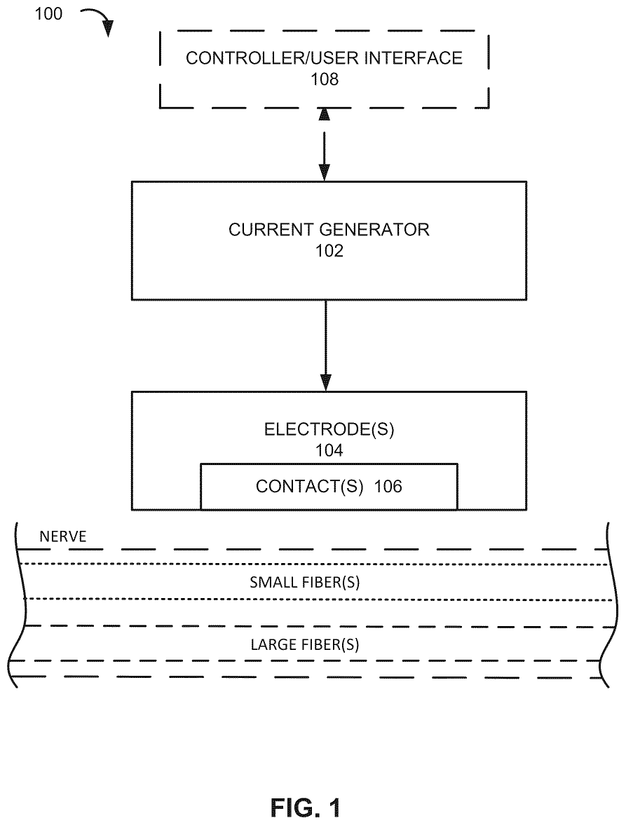

FIG. 1 shows a block diagram of a system 100 that can perform focused direct current neuromodulation on at least one nerve enabling a small-diameter and/or unmyelinated fiber selective block (the opposite of the electrical order of recruitment). The system 100 can include a current generator 102 in electrical communication (e.g., generally wired) with at least one electrode (e.g., electrode(s) 104) having at least one contact (e.g., contact(s) 106). The electrode(s) 104 can be positioned near, adjacent, and/or in direct contact with at least one nerve. The electrode(s) 104 can be implanted and/or external on the skin surface, and can be removably positioned, and/or held in position for a time of application depending on the circumstance and the condition to be treated (e.g., pain, chronic pain, spasticity, autonomic dysregulation, etc.). The current generator 102 can be external to the patient, percutaneous, and/or implanted in the patient.

The current generator 102 can be any device configured or programmed to generate the at least one direct current for application to a nerve to achieve an alternation in conduction of at least one small-diameter nerve fiber thereof. The current generator 102 can generate a direct current with an amplitude (e.g., 15 mA) for a time. The direct current can have an amplitude of 30 mA or less, 25 mA or less, 20 mA or less, 15 mA or less, 10 mA or less, 1 mA or less, or the like. It should be understood that the amplitude of the direct current, when generated, is always non-zero. The time can be from 10 ms to 5 hours, from 1 second to 3 hours, from 1 minute to 1 hour, 30 minutes plus or minus 10 minutes, 2 minutes plus or minus 1.5 minutes, or the like. For instance, the time can be 30 minutes or less and the at least partial block of the smaller-diameter nerve fibers can last four hours or less after the application of the direct current has ended (e.g., for pain relief). In another instance, the time can be 30 minutes (plus or minus ten minutes) and the direct current can be applied in five minute bursts over the 30 minutes (plus or minus ten minutes). Such an application can be repeated up to four or five times a day (e.g., to regulate an autonomic dysfunction—such as bladder regulation). In a further instance, the time can be from 1 minute to 2 minutes (plus or minus 30 seconds) and the application can be repeated a plurality of times a day (e.g., from 1 to 20 times, from 1 to 10 times, or the like) (e.g., for spasticity relief).

One example of a current generator 102 can be a battery-powered, portable generator. Another example of a current generator 102 can be an implantable generator (IPG). It should be appreciated that the current generator 102 can include additional components to selectively configure the current waveform, such as an amplitude modulator (not shown). While a monophasic direct current is discussed throughout, it should be understood that the current generator 102 can be configured or programmed to generate a direct current having a monophasic waveform or a biphasic or a substantially bi-phasic waveform with one phase cathodic and another phase anodic. The current generator 102 can reverse the polarity of the direct current as needed to reduce the chances of nerve damage and at least partially balance charge with the biphasic or substantially biphasic waveform. It should be understood that the monophasic waveform can include different amplitudes at different times (e.g., increasing or decreasing the affects). The current generator 102 can send the current to one or more of the electrode(s) 104.

The electrode(s) 104 via contact(s) 106 can apply the at least one direct current generated by current generator 102 to the nerve for the time (e.g., the time of generation and application can be the same). For instance, one electrode 104 and/or multiple electrodes can provide direct current(s) to a single nerve. In another instance, one electrode 104 can provide a direct current to multiple nerve(s) if the nerves are in close enough proximity to each other and the electrode. In a further instance multiple electrodes 104 can be positioned such that each electrode can apply direct current to a different designated nerve of a plurality of nerves (and the direct currents can be different per nerve). Each target nerve can include at least one small-diameter nerve fiber (unmyelinated or thinly myelinated) and at least one large-diameter, myelinated nerve fiber.

The electrode(s) 104 can be of any form and/or composition that can safely provide a direct current without causing significant harm to a nerve (e.g., via reaction products). The electrode(s) 104 form and/or composition can be a separated nerve interface electrode, an electrode at least partially composed of or coated with a high charge capacity material (e.g., specifically designed with a shape and/or material to store a large amount of electrical charge), or the like. In non-exhaustive list, the electrode(s) 104 can be at least one of a focused tripolar electrode with at least three contacts, a nerve cuff electrode with at least one contact, a SINE electrode with at least one contact, and/or a percutaneous electrode with at least one contact. If the electrode(s) 104 is a focused tripolar electrode then a center contact can be cathodic and the flanking contacts can be anodic. The application of the direct current to the nerve for the time can create a spatially restricted direct field based on the direct current's parameters and the electrode(s) 104 configuration and design. The spatially restricted direct current field can be configured (e.g., by the generator, the electrode design and configuration, etc.) to at least partially block conduction in small-diameter nerve fibers of the nerve while leaving large-diameter, myelinated nerve fibers of the nerve able to conduct.

The contact(s) 106 of the electrode(s) 104 can apply (directly or indirectly) the direct current with an amplitude for a time to at least one nerve. Application of the direct current can cause the generation of a direct current field based on at least the amplitude and the time. In this case, the field can be a spatially restricted direct current field. The spatially restricted direct current field can be a focused field that can extend along a length of the nerve that is thinner than a traditional broad direct current field so that the spatially restricted direct current field can reach a smaller number of nodes of Ranvier in a large-diameter, myelinated nerve fibers than a traditional broad direct current field. For instance, the focused field can extend along the length of the nerve and be smaller and more focused than a standard field created by traditional neuromodulation (e.g., the focused field can extend a distance that is smaller/thinner than a distance between 5 nodes of Ranvier in the large-diameter, myelinated nerve fiber(s)). As an example, the direct current can be applied to at least one sensory and/or sensorimotor nerve to selectively inhibit conduction in at least one small-diameter nerve fiber (e.g., C-fibers, Aδ-fibers, or the like) without affecting, or minimally affecting, conduction of at least one large-diameter, myelinated nerve fiber(s) (e.g., Aα and/or Aβ fibers) in the same nerve. For instance, the focused direct current field generated can block conduction of pain signals, while allowing (most or all) of conduction of voluntary muscle movement signals. For example, when the focused direct current field causes pain relief, the pain relief effect can last minutes to hours after application of the direct current has ended.

The system 100 can also, in some instances, include a controller/user interface 108 (optional). The controller/user interface can be in communication (wired and/or wireless) with the current generator 102. The controller/user interface 108 can be at least external to the patient and can be operated by a medical professional and/or the patient (e.g., with safety limitations in place based on a prescription). It should be understood that the controller can include the user interface, but needs not include the user interface.

In some instances, the controller/user interface 108 can include a non-transitory memory and a processor (not illustrated in FIG. 1) and a user interface that can be used (by a medical professional and/or a patient) to review, input, and/or re-configure at least one parameter of the at least one direct current generated by the current generator 102. The at least one parameter can be amplitude, time of application, voltage, resistance, power, or the like. As an example, the memory and processor can be embodied as a microprocessor. The parameters can be determined/chosen based on at least one of the condition to be treated, the location of the target nerve, the type of target fiber(s), electrode configuration details, the amount of relief, the length of relief, the patient's body conditions (e.g., health, weight, age, gender, muscle mass, etc.), or the like. The user interface of the controller/user interface 108 can include at least one of a visual display, a speaker, an input mechanism (e.g., touch screen, keyboard, a microphone, or the like), or the like. The controller/user interface 108 can be, for instance, a mobile phone, a tablet, a computer, a designated external controller, or the like. Alternatively, the controller/user interface 108 and the current generator 102 can be embodied within a single device that is at least partially external to the patient.

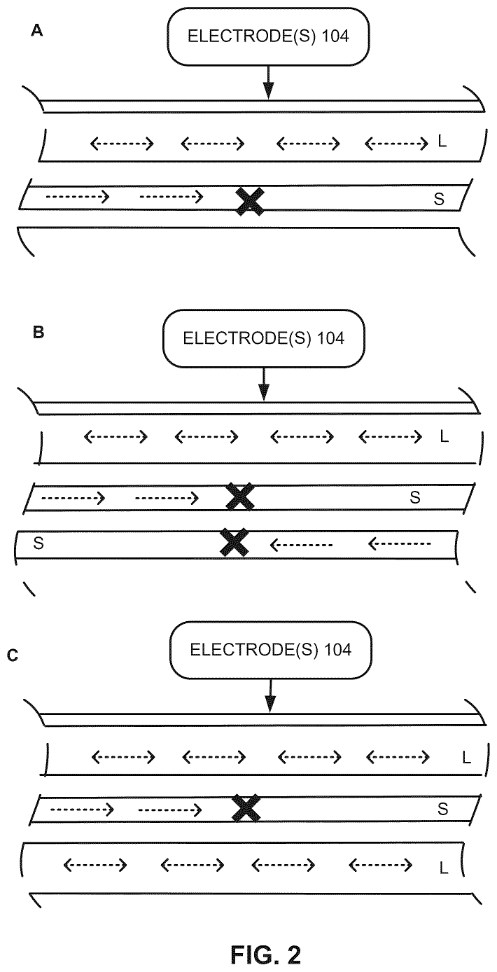

FIG. 2 shows examples of using the system of FIG. 1 to apply focused direct current neuromodulation to generate a spatially focused direct current field that blocks conduction in at least one small-diameter nerve fiber in at least one nerve while not affecting, or, in some instances, minimally affecting, conduction in at least one large-diameter, myelinated nerve fiber of the same nerve(s). As illustrated, conduction in large-diameter, myelinated nerve fibers can be in either an upstream or downstream directions, such that the large-diameter, myelinated nerve fibers can be motor fibers (conducting mechanical signals to muscles) and/or large sensory fibers. Generally conduction in small fiber(s) is uni-directional depending on fiber (as shown, the conduction can proceed in either direction depending on the type of fiber), but if physiologically possible may be bi-directional. It should be understood that a given nerve can have multiple nerve fibers (of varying types/sizes and numbers per type/size) that can each conduct in varying directions; the two-headed arrows are shown in FIG. 2 to signify uni-directional conduction in either direction in one nerve (depending on nerve type and location) and/or, if physiologically possible, bi-directional conduction in a single fiber.

Several examples of fiber selective focused direct current neuromodulation are shown in FIG. 2 for illustrative purposes only and are not intended to be limiting. FIG. 2, element A illustrates the electrode 104 being positioned nearer a large-diameter, myelinated nerve fiber (L) than a small-diameter nerve fiber(S) and blocking conduction in the small-diameter nerve fiber but not in the large-diameter, myelinated nerve fiber. FIG. 2, element B illustrates the electrode(s) 104 positioned relative a nerve with two small-diameter nerve fibers (shown as conducting in different directions but may conduct in the same direction) and one large-diameter, myelinated nerve fiber, where conduction in both small-diameter nerve fibers are blocked and conduction in the large-diameter, myelinated nerve fiber is not. FIG. 2, element C illustrates the electrode(s) 104 positioned relative a nerve with a small-diameter nerve fiber between two large-diameter, myelinated nerve fibers and conduction in both large-diameter, myelinated nerve fibers is not affected while conduction in the middle small-diameter nerve fiber is blocked.

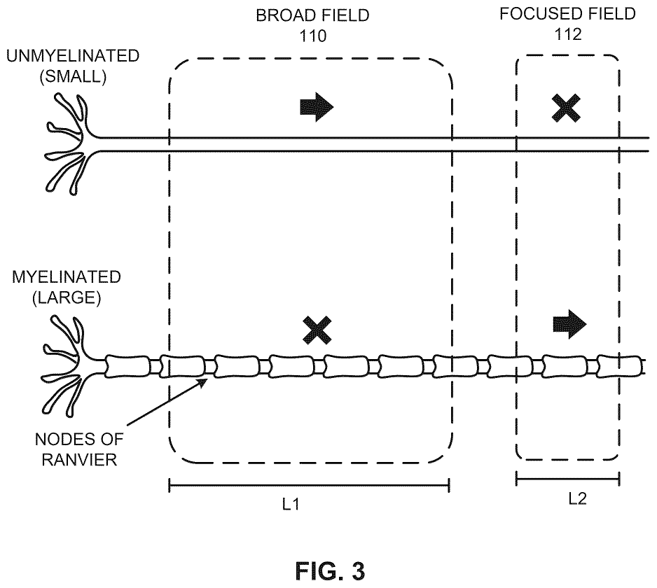

FIG. 3 shows an illustration of the biological mechanism behind focused direct current neuromodulation selectively inhibiting conduction in at least one small-diameter nerve fiber (e.g., C-fibers, Ad-fibers, or the like) (or in some instances, unmyelinated fibers) without affecting, or minimally affecting, conduction of at least one large-diameter, myelinated nerve fiber(s) (e.g., Aα and/or Aβ fibers) in the same nerve. FIG. 3 also compares the focused field 112 that can be produced by system 100 and the traditional broad field 110 that is applied for traditional neuromodulation therapies. The spatially restricted direct current field can be a focused field (e.g., focused field 112) that can extend along a length L2 of the nerve that is thinner (shorter) than a length of a nerve L1 that a traditional broad direct current field (e.g., broad field 110) can extend along. As an example, the spatially restricted direct current field (e.g., focused field 112) can reach a smaller number of nodes of Ranvier (e.g., less than 4-5) in a large-diameter, myelinated nerve fibers than the traditional broad field (broad field 110).

The broad field 110 can block conduction in the large-diameter, myelinated nerve fiber, but may not block conduction in the small-diameter and/or unmyelinated nerve fiber (depending on the amplitude and/or time of application of the DC the broad field may also block conduction in the small-diameter nerve fiber). The focused field 112 can block conduction in the small-diameter and/or unmyelinated nerve fiber, while still allowing conduction in the large-diameter, myelinated nerve fiber. This can be based on the physical differences in large-diameter and/or myelinated nerve fibers.

As an example, the myelinated nerve fiber propagates with saltatory conduction, while unmyelinated fibers propagate with continuous conduction. In saltatory conduction, action potentials are generated at each of the nodes of Ranvier, which have a high concentration of sodium potassium channels. Inside the myelinated portion of the large diameter, myelinated nerve fiber, a signal (e.g., an electrical signal) rapidly propagates to the next node where the signal generates another action potential. In myelinated nerve fibers, 4-5 nodes of Ranvier need to be inactivated to prevent action potential propagation (e.g., form a block in the myelinated nerve fibers). Large-diameter, myelinated fibers (Aα and Aβ) also have larger internodal distances making it possible to inactivate less than 4-5 nodes of Ranvier with a focused field that can still block small-diameter nerve fibers (and/or unmyelinated nerve fibers). Focused direct current neuromodulation creates a depolarized region in nerve fibers that is narrower than the span of the 4-5 nodes of Ranvier needed to block large-diameter, myelinated nerve fibers while blocking unmyelinated and/or small-diameter nerve fibers that have continuous conduction as well as slow conduction velocities. Therefore, a spatially restricted DC field that inactivates 4-5 nodes of a smaller diameter nerve fiber (nociceptive Aδ) will prevent conduction in the Aδ yet will not affect the minimum nodes to block conduction in the Aα and Aβ fibers. Combined, this results in nociceptive nerve fiber (C and Aδ) block at lower amplitudes, providing a clinically advantageous nociceptive-selective nerve block (and also works for other small diameter and/or unmyelinated nerve fibers).

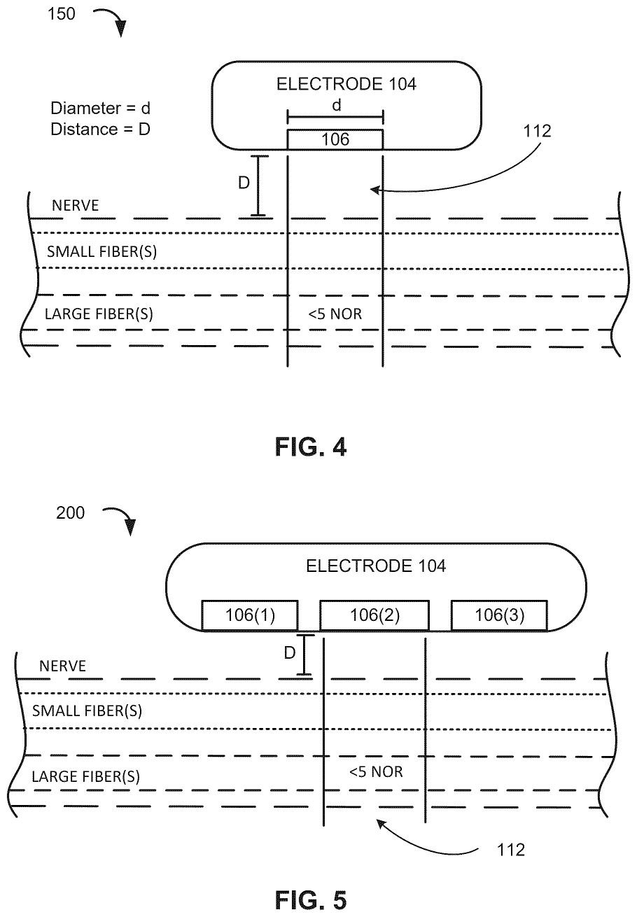

FIG. 4 and FIG. 5 show illustrations of different example electrode configurations that can form the focused field 112 for selective fiber block based on application of a direct current to a nerve. FIG. 4 shows an example 150 of an electrode 104 (of system 100) that has a single contact 106 applying the direct current to cause a focused direct current field to be applied to a nerve containing both small-diameter and/or unmyelinated nerve fibers and large-diameter, myelinated nerve fibers. The diameter (d) of the contact 106 and the distance (D) of the contact from the nerve can inform how focused the focused field 112 is in a given direct current application. The diameter of the contact 106 can be 1.5 mm or less, 1 mm or less, 0.26 mm or less, or the like. Generally, the smaller the diameter of contact 106 the further away (distance, D) from the nerve the electrode 104 can be positioned/held/implanted.

FIG. 5 shows an example 200 of an electrode 104 (of system 100) that is a tripolar electrode (e.g., three contacts 106(1), (2), and (3)) applying a focused direct current to a nerve containing both small-diameter and/or unmyelinated nerve fibers and large-diameter, myelinated nerve fibers. The tripolar electrode 104 can deliver the direct current and shape the direct current to focus the focused field 112 without requiring the geometric size (e.g., diameter) size constraints of FIG. 4. The central contact 106(2) can be cathodic and the flanking contacts 106(1) and (2) can be anodic. The contacts 106(1), (2), and (3) can be from 0.1 mm to 2 μm, about 1 mm to 2 mm in diameter, or the like, and can have a distance between them from 0.1 mm to 2 mm, from 1 mm to 2 mm, or the like. Without wishing to be bound by theory, the smaller and closer together the tripolar electrode contacts can be the more focused the field can be and the further away from the nerve the electrode 104 (distance D) can be positioned/held/implanted.

Without wishing to be bound by theory, fiber specific block can be achieved with an electrode contact 106 tip with a diameter of 0.26 mm and was NOT achieved at 1.75 mm. However, contact diameter size can depend on the distance of the tip of the contact 106 from the nerve. As the tip gets farther away, the field that reaches the nerve expands. So the further the electrode is positioned from the nerve, the smaller the diameter of electrode contact tip would be needed to get the same effect. Without wishing to be bound by theory, to block a large-diameter, myelinated nerve, 4-5 nodes of Ranvier need to be blocked. The nodes of Ranvier are generally 1 μm-2 μm in length but are separated by internodes that are proportional to the fiber size with the internode being 100-150× the axon diameter. Aα fibers are 13-20 μm in diameter, so the minimum field it would take to block them would be 13 μm×100×4 nodes=5.2 mm and the maximum 20 μm×150×5 =15.0 mm. Aβ fibers are 6-12 μm in diameter, so the minimum field it would take to block them would be 6μm×100×4 nodes=2.4 mm and the maximum 12 μm×150×5 =9.0 mm. Aδ fibers are 1-5μm in diameter, so the minimum field it would take to block them would be 1 μm×100×4 nodes=0.4 mm and the maximum 5 μm×150×5 =3.75 mm. The C fibers have no myelin. Therefore, the length that would need to be blocked, to get just C fibers would be 0.4<x<3.75 mm. To get a block of both C and Aδ the focused field would be between 2.4 mm<x<9.0 mm.

### IV. Methods

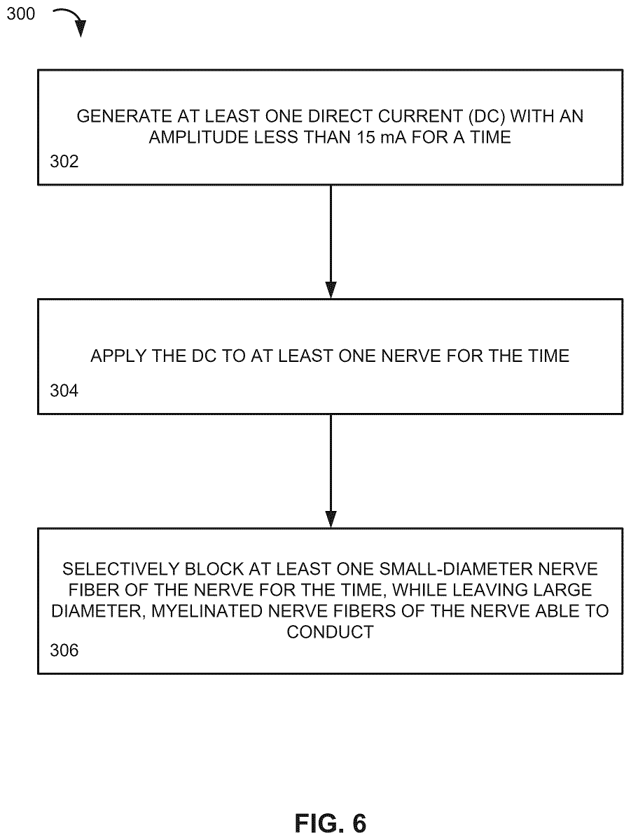

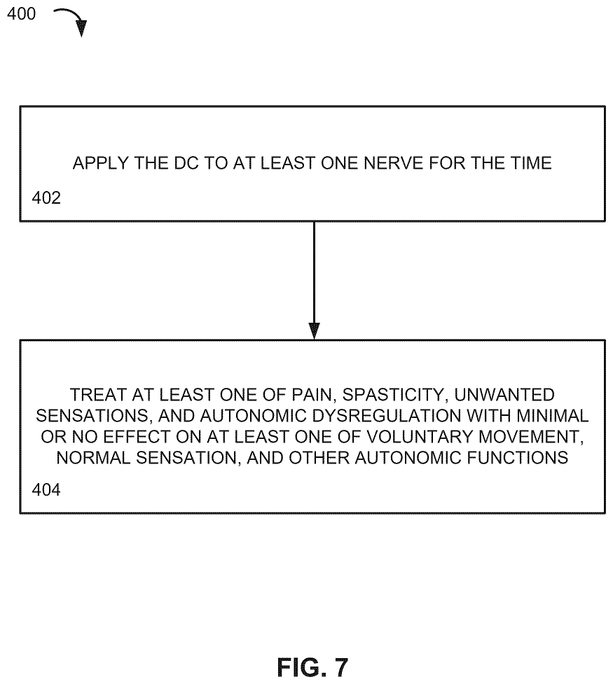

Another aspect of the present disclosure can include methods for focused direct current neuromodulation 300 and for treating one or more conditions (e.g., pain, spasticity, autonomic dysregulation, etc.) with focused direct current neuromodulation 400 (e.g., with the system of FIGS. 1-5). The methods 300 and 400 are illustrated as processes flow diagrams with flowchart illustrations that can be implemented by one or more components of the system, as shown in FIG. 1. For purposes of simplicity, the methods 300 and 400 of FIGS. 6 and 7 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement methods 300 and 400.

FIG. 6 shows a method 600 for performing focused direct current neuromodulation to selectively block at least one small-diameter fiber in a nerve. The method 300 can bock conduction in at least one small-diameter nerve fiber while leaving at least one large-diameter, myelinated nerve fiber in the same nerve at least partially unblocked and able to conduct. While not shown, the patient can undergo a surgery or other procedure to position at least one electrode (e.g., electrode(s) 104) near, adjacent, and/or in direct contact with at least one target nerve. The electrode(s) can be implanted, removably positioned, and/or held in position for a time of application depending on the circumstance and the condition to be treated. At 302, at least one direct current (DC) can be generated (e.g., by a current generator 102 in electrical communication with the electrode(s) 104). The direct current can be generated for a time (e.g., from 10 ms to 5 hours, from 1 second to 3 hours, from 1 minute to 1 hour, 30 minutes plus or minus 10 minutes, 2 minutes plus or minus 1.5 minutes, or the like). The direct current can have an amplitude of 30 mA or less, 25 mA or less, 20 mA or less, 15 mA or less, 10 mA or less, or the like. It should be understood that the amplitude of the direct current, when generated, is always non-zero.

At 304, the at least one direct current can be applied to the at least one nerve by the at least one electrode (e.g., electrode(s) 104) for the time. The application of the direct current for the time forms a spatially restricted (e.g., focused) DC field. The spatially restricted DC field can be a focused field that has a thinner length (e.g., along the length of the nerve) than a traditional broad DC field. For instance, the focused field can be thinner than a distance between 4 or 5 (plus or minus 1) nodes of Ranvier in the large-diameter, myelinated nerve fiber(s). The size of the spatially restricted DC field can be based on parameters of the direct current, the electrode(s) design and configuration, and/or the distance the electrode(s) is from the nerve(s).

For instance, one electrode (e.g., 104 of system 100) that has a single contact (e.g., 106) can apply the direct current to cause a focused direct current field to a nerve containing both small-diameter and/or unmyelinated nerve fibers and large-diameter, myelinated nerve fibers. The diameter (d) of the contact 106 and the distance (D) of the contact from the nerve can inform how focused the focused field (e.g., 112) is in a given direct current application. The diameter of the contact 106 can be 1.5 mm or less, 1 mm or less, 0.26 mm or less, or the like to focus the field smaller than the distance of the amount of nodes of Ranvier necessary to block conduction in the large-diameter, myelinated fibers (e.g., less than 4 or 5 nodes of Ranvier). Generally, the smaller the diameter of contact the further away (distance, D) from the nerve the electrode can be positioned/held/implanted.

In another instance, one electrode (e.g., 104 of system 100) that is a tripolar electrode (e.g., has three contacts 106(1), (2), and (3)) can apply a focused direct current to a nerve containing both small-diameter and/or unmyelinated nerve fibers and large-diameter, myelinated nerve fibers. The tripolar electrode can deliver the direct current and shape the direct current to focus the focused field (e.g., 112, thinner than 4 or 5 nodes of Ranvier on the large-diameter, myelinated nerve fibers) without requiring the geometric size (e.g., diameter) size constraints of FIG. 4. The central contact 106(2) can be cathodic and the flanking contacts 106(1) and (2) can be anodic. The contacts 106(1), (2), and (3) can be from 0.1 mm to 2 mm, about 1 mm to 2 mm in diameter, or the like, and can have a distance between them from 0.1 mm to 2 mm, from 1 mm to 2 mm, or the like. Without wishing to be bound by theory, the smaller and closer together the tripolar electrode contacts can be the more focused the field can be and the further away from the nerve the electrode 104 (distance D) can be positioned/held/implanted

At 306, the at least one small-diameter nerve fiber (and/or unmyelinated nerve fiber) of the at least one nerve can be at least partially selectively blocked by the application of the at least one direct current, while leaving at least one large diameter, myelinated nerve fiber of the same nerve(s) able to conduct. The selective block can be partial or full block of the at least one small-diameter nerve fiber depending on the configurations of the DC and desired outcome. For instance, the conduction that can be blocked in the small-diameter nerve fibers can be conduction that cause neuropathic pain to be felt by the patient. While the neuropathic pain feeling is at least partially blocked, conduction in the large-diameter, myelinated nerve fibers in the same nerve(s) can at least partially continue (e.g., in an unaffected or minimally affected manner). In another instance, the conduction that can be blocked in the small-diameter nerve fibers can be conduction that causes spasticity in at least one muscle of the patient. While the spasticity is at least partially blocked, conduction in the large-diameter, myelinated nerve fibers in the same nerve(s) can at least partially continue (e.g., in an unaffected or minimally affected manner). In a further instance, the conduction that can be blocked in the small-diameter nerve fibers can be conduction that causes some amount of autonomic dysregulation. While the autonomic dysregulation signals are at least partially blocked, conduction in the large-diameter, myelinated nerve fibers in the same nerve(s) can at least partially continue (e.g., in an unaffected or minimally affected manner).

FIG. 7 shows a method 400 for treating at least one condition (e.g., pain, spasticity, an autonomic dysregulation, or the like). At 402, the direct current can be applied to the at least one nerve for a time and at an amplitude as discussed above (e.g., via electrode(s) 104 and current generator 102) to provide a focused DC field. At 404, at least one of pain, spasticity, unwanted sensations, and autonomic dysregulation with minimal or no effect on at least one of voluntary movement, normal sensation and other autonomic functions (e.g., normal or not intended to be treated functions). For example, to at least partially block pain, while allowing normal or significantly normal (e.g., at least 80%-99% of conduction) conduction in large-diameter nerve fibers (e.g., to continue normal or nearly normal voluntary movement), the focused direct current neuromodulation can be applied in one or more 30 minute increments (plus or minus 10 minutes). The pain relief effect can last for 30 minutes to 5 hours after the time of application of the focused direct current neuromodulation. In another example, to at least partially block an autonomic nerve causing autonomic dysregulation, while allowing normal or significantly normal (e.g., at least 80%-99% of conduction) conduction in large-diameter nerve fibers, the focused direct current neuromodulation can be applied in burst patterns one or more times a day (e.g., bursts of 5 minutes 4 times a day, depending on the autonomic function/dysregulation). In a further example, to at least partially control spasticity, while allowing normal or significantly normal (e.g., at least 80%-99% of conduction) conduction in large-diameter nerve fibers (e.g., to continue normal or nearly normal voluntary movement), the focused direct current neuromodulation can be applied in for about 30 seconds to 5 minutes, 1 minute to 2 minutes, or the like between 1 and 20 times a day (e.g., for 1-2 minutes 10 times a day).

### V. Clinical Applications

The ability to specifically select fiber diameters to block, can make possible clinical applications that previously were not feasible due to the simultaneous block of large-diameter, myelinated fibers (including motor fibers) with small-diameter and/or unmyelinated fiber block.

For instance, in the sensory system, chronic neuropathic pain can be treated with fiber specific focused direct current neuromodulation. For example, Knee Osteoarthritis (KOA) is a progressive disease that effects more than 30% of older adults resulting in chronic pain, functional disability, reduced quality of life, and decline in physical and mental health. Conventional KOA pain treatment varies throughout disease progression, starting with conservative treatments such as weight loss, physical therapy, anti-inflammatory medications, and intra-articular steroid injections to retain mobility and relieve pain. If conservative treatments are not adequate, more advanced treatments such as genicular nerve block (GNB), radiofrequency ablation (RFA) and peripheral nerve stimulation (PNS) are implemented. If conservative and more advanced treatments fail to provide full analgesia, the individual may need to undergo total knee arthroplasty (TKA). However, even in those who undergo TKA, 25% have chronic persistent postsurgical pain. Analysis of clinical outcomes suggests that one possible source of chronic post-surgical pain is the presence of unrelieved pain pre-surgery. Fiber specific focused direct current neuromodulation can be applied (upon application of a DC) to at least one nerve of a patient to at least partially relieve pain pre surgery. It is also noted that, effective control of post-operative pain can prevent the development of chronic pain by reducing peripheral and central sensitization. Chronic pain is a complicated, difficult-to-treat condition that involves central pathways, psychosocial factors as well as afferent sensitization. Fiber specific focused direct current neuromodulation can be applied to at least one nerve of a patient to at least partially relieve, stop, and/or reverse the development of chronic pain.

In addition to pain relief, rehabilitation goals for degenerative joint disease can include restoring strength and range of motion so that an individual can return to activities of daily living. To achieve this, there are two main clinical needs that must be addressed: adequate pain control and the functional ability to perform therapeutic exercises. For individuals who prefer non-operative treatment, or those who are nonoperative candidates, pain control is necessary to maintain compliance with physical therapy and home exercises that have been shown to results in better outcomes. To address these shortcomings, fiber specific neuromodulation provides a fast acting, adjustable, and reversible small-diameter nerve fiber pain blockade of femoral and sciatic nerves without affecting motor fibers (e.g., large-diameter, myelinated nerve fibers) and motor function. This approach can provide a single solution that can be used in all phases of the disease progression thereby reducing costs and eliminating the learning curve for the user.

Fiber specific focused direct current neuromodulation can be adjusted as necessary to manage ongoing pain while allowing the patient to continue physical therapy and activities of daily living. Control of a pain blockade can be controlled in real time (e.g., via controller/user interface 108 and/or changing location). With this technology, complete pain blockade can be provided continuously, while allowing for decremented pain relief that can allow individuals and their clinicians to track the progression of pain intensity over time. Additionally, pain relief can be appropriately titrated so that the fiber specific focused direct current neuromodulation can maintain a protective effect without chronic pain progressing to a chronic pain syndrome with central sensitization.

In another instance, in the autonomic system, there are several different mixed nerves that can be more finely controlled with fiber specific focused direct current neuromodulation. Specifically, the vagus nerve controls a wide range of functions depending on the location of the electrode and contains both afferent and efferent nerves. Vagal nerve stimulation is often accompanied by unwanted side effects due to non-specific activation of nerves. Fiber specific focused direct current neuromodulation can provide specific block of sensory nerves to prevent off target side effects. The pudendal nerve also carries both sensory information as well as motor innervation to the sphincter. A fiber specific block of the pudendal nerve can provide a more targeted bladder control system compared to current methods. Further, in renal denervation (RDN) for hypertension both afferent and efferent pathways are targeted. The use of fiber specific focused direct current neuromodulation can provide more patient specific intervention that can be controlled in a closed loop paradigm.

In a further instance, in the motor system, stroke is a debilitating condition that results in many different neurological challenges. Electrical stimulation has been used as a treatment method for restoring hand function in stroke. However, the presence of spasticity in stroke has limited the types of patients that can be helped by current electrical therapies. Spasticity can also be a component of many different neurological conditions such as cerebral palsy, multiple sclerosis, spinal cord injury, in addition to stroke. The onset of spasticity related to any of these illnesses/conditions results in many impairments and limitations such as gait disorders, fatigue, restricted range of movement, abnormal limb postures, quality of life issues, problems with activities of daily living, and pain. Fiber specific focused direct current neuromodulation can address the sensory sources of spasticity while allowing for continued motor function.

### VI. Experimental

The following experiments demonstrate fiber selectivity with focused direct current neuromodulation. The first part of the experiment was an acute in vivo experimental setup that allowed researchers to collect two types of sensory electrophysiological signal in the same animal. The second part of the experiment included computational modeling of another electrode design and configuration for focused direct current neuromodulation.

### 1. First Experiment—Geometric Embodiment

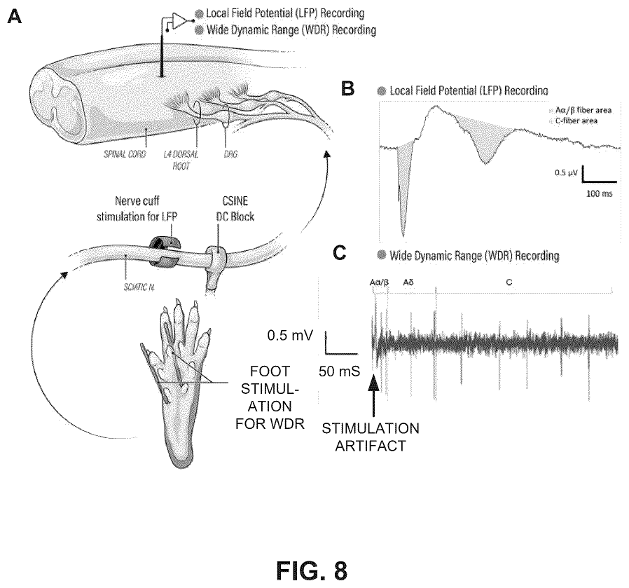

To evaluate the efficacy of block, sensory nerves were activated by stimulation of the plantar region of the hind paw (electrical or heat) or via direct electrical stimulation of the sciatic nerve (see FIG. 8). For each modality, the recordings were discriminated by activation threshold and latency.

FIG. 8, element A shows the experimental setup for the acute experiments. The pain signals are elicited either in the foot (with needle electrode stimulation) or via direct stimulation of the sciatic nerve. The fiber selective-focused direct current neuromodulation (FS-FDCN) electrode is placed proximally to the stimulating electrode. Recordings in the spinal cord were made of either the local field potentials (LFP) or of a single neuron that receives input from a wide gamut of fiber sizes, including both unmyelinated and myelinated fibers, called a wide dynamic range (WDR) neuron.

FIG. 8, element B shows a graphical representation of local field potentials (LFP) recorded from the spinal dorsal columns using high impedance (1 MΩ) microelectrodes. The high impedance microelectrodes were used to measure the activation of the whole sciatic nerve; the LFP represented the responses of many cells, including excitatory postsynaptic potentials in the dendrites and action potentials recorded from the soma and axon terminals. FIG. 8, element C shows a graphical representation of wide dynamic range (WDR) recordings. WDR neurons are present in the dorsal horn of the L4 and L5 spinal cord. These neurons are multi-receptive; they receive, and faithfully transmit, input from all subclasses of sensory neuron: nociceptive Aδ- and C-fibers, and non-nociceptive Aα/β-fibers. Therefore, based on the latency with respect to the sensory stimulus, the activity of WDR neurons provided detailed information about peripheral sensory activity.

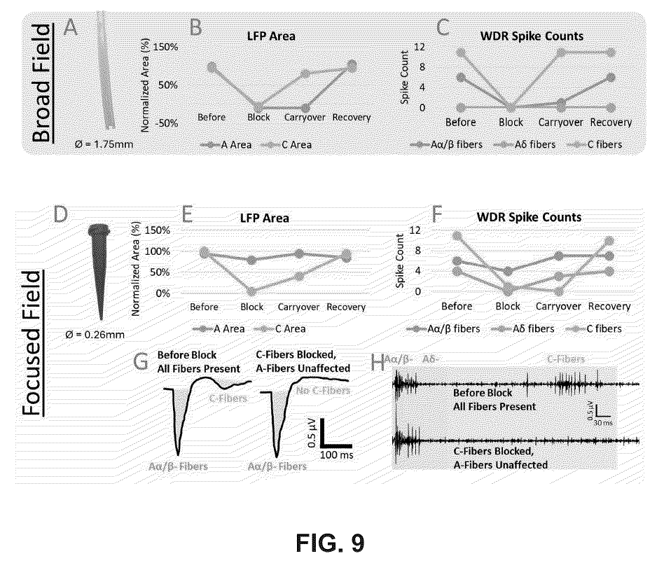

Summaries from preliminary trials can be seen in FIG. 9 for both LFP and WDR. FIG. 9, element A shows the electrode used for the ‘broad’ field block: a 1.75 mm inner diameter tube with a notch to hold the nerve. When a blocking current was applied that was sufficient to block C-fibers, it was seen that A-fibers were also blocked in both the LFP (see FIG. 9, element B) and WDR (see FIG. 9, element C) experiments. FIG. 9, element D shows the electrode used for the focused monopolar fields: a 25 Ga. dispensing needle with a 0.26 mm opening at the tip. With the focused field, complete block of C-fibers were seen without blocking A-fibers in the LFP signal (see FIG. 9, element E). In the WDR recordings (see FIG. 9, element F), there was some attenuation of the Aα/β- and Aδ-fibers with block, but immediately after the current was switched off, the C-fibers remained blocked, while the Aα/β- and Aδ-fibers recovered immediately. FIG. 9, element G shows an example of selective C-fiber block in LFP experiments, and similarly an example of selective C-fiber block is seen in WDR recordings in FIG. 9, element H.

The experiments compared the block selectivity between 2 different monopolar nerve interfaces that created either a broad (see FIG. 9, elements A-C) or spatially focused (see FIG. 9, elements D-H) DC field. A focused DC field was achieved by using an electrode tip with a smaller aperture (0.26 mm) vs a larger aperture (1.75 mm). The tip was placed directly on the nerve to provide nerve block using the CSINE device. In anesthetized rats, sensory stimulation was provided using either a foot electrode (WDR) or a cuff electrode placed distal to the block electrode (LFP). The stimulation levels were chosen to elicit both A-and C-fiber response which can be detected in both the LFP and WDR measurements. Data was collected during four separate experiments (2 electrode types×2 recording techniques). After recording a baseline response to stimulation, the block was turned on, and the change in A-and C-fiber activation was recorded during induction of sensory nerve block and until recovery to baseline activity. DC amplitudes were adjusted in successive trials until a C-fiber block was observed. Selective block was seen both while the block was on, and for a duration after the block was turned off (see FIG. 9, elements E and F).

### 2. Second Experiment—Waveform Shaping Embodiment

Although the in vivo testing demonstrated that a narrow field produced by a small tip CSINE electrode can produce selective block, this embodiment was shown to have a high impedance and was tested on an ideal location directly touching the nerve. It is possible to shape the DC field using alternative electrode configurations such as a tripolar, where flanking anodic regions produce a focused central cathodic field. This allowed the customization of the electrode geometry to minimize device constraints as well as to allow a more robust interface in terms of electrode proximity.

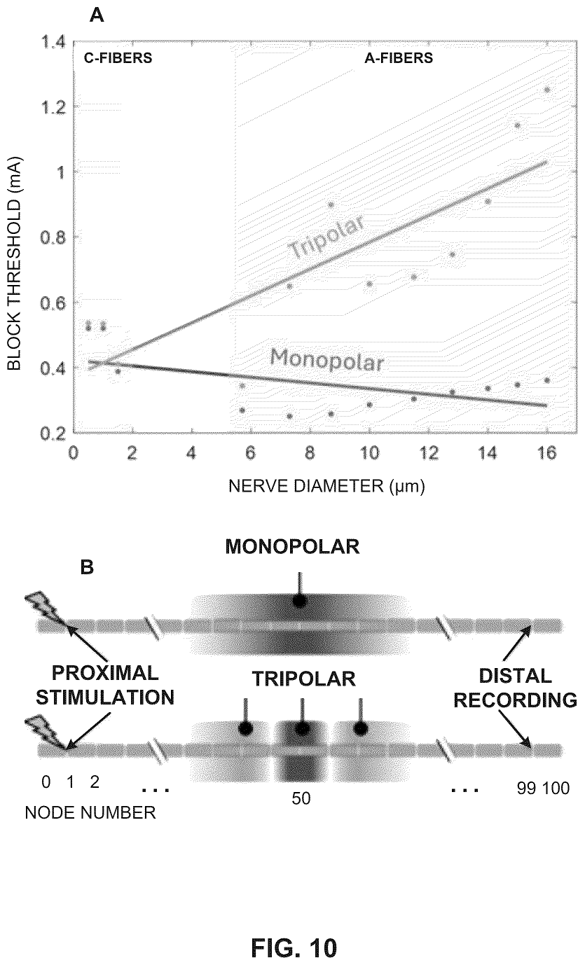

To test the efficacy of the focused tripolar electrode, a modeling study was performed to compare the focused tripolar to a typical broad monopolar electrode (see FIG. 10 element B). The simulations were run in the NEURON environment (version 8.2.0). The experiments were designed to compare the block thresholds (the minimum amplitude needed to achieve complete block) for both electrode types in various sizes of myelinated and unmyelinated fibers. The electrodes were simulated using point sources 1 mm from the simulated axon. This 1 mm is farther from the nerve than seen in in vivo experiments; was not meant to represent an electrode that far away from the axon, but to emulate the field spread caused by a wider electrode that would be closer to the axon. The broad monopolar electrode was a single point source directly in the middle of the axon directly above the middle Node of Ranvier. The focused tripolar had the same source in the middle of the axon, but also two flanking sources each 1 mm away from the central ‘electrode’ and 1 mm away from the axon (see FIG. 10, element B). The results of this study show that the focused tripolar electrodes block C-fibers preferentially to A-fibers, whereas the broad monopolar electrode preferentially blocks A-fibers at lower amplitudes to C-fibers (see FIG. 10, element A). With similar block thresholds for the C-fibers, tripolar electrodes appears to create its selectivity not by improving C-fiber block, but by impairing A-fiber block, thereby increasing A-fiber block amplitude.

From the above description, those skilled in the art will perceive improvements, changes and modifications. Such improvements, changes and modifications are within the skill of one in the art and are intended to be covered by the appended claims.

What is claimed is:

1. A system comprising:
a current generator configured to generate at least one direct current (DC) with an amplitude of less than 15 mA for a time; and
at least one electrode coupled to the current generator and configured to apply the DC to a nerve for the time,
wherein application of the DC to the nerve for the time creates a spatially restricted DC field based on the DC and the at least one electrode, such that the spatially restricted DC field is configured to at least partially block conduction in small-diameter nerve fibers of the nerve while leaving large-diameter, myelinated nerve fibers of the nerve able to conduct.

2. The system of claim 1, wherein the spatially restricted DC field is a focused field that extends along a length of the nerve that is thinner than a traditional broad DC field so that the spatially restricted DC field reaches a smaller number of nodes of Ranvier in a large-diameter, myelinated nerve fibers than a traditional broad DC field.

3. The system of claim 2, wherein the focused field extends along the length of the nerve that is thinner than a distance between 5 nodes of Ranvier in the large-diameter, myelinated nerve fibers.

4. The system of claim 1, wherein the small-diameter nerve fibers are unmyelinated C fibers and the large-diameter, myelinated nerve fibers are myelinated Aα and/or Aβ fibers.

5. The system of claim 1, wherein the small-diameter nerve fibers are myelinated Ad fibers and the large-diameter, myelinated nerve fibers are myelinated Aα and/or Aβ fibers.

6. The system of claim 1, wherein the at least one electrode comprises one or more contacts, each with a diameter less than 1.75 mm.

7. The system of claim 1, wherein the at least one electrode comprises one or more contacts, each with a diameter of 1 mm or less.

8. The system of claim 1, wherein the at least one electrode comprises one or more contacts, each with a diameter of 0.2 mm or more.

9. The system of claim 1, wherein the at least one electrode is a focused tripolar electrode with at least three contacts, a nerve cuff electrode with at least one contact, a SINE electrode with at least one contact, and/or a percutaneous electrode with at least one contact.

10. The system of claim 1, wherein the at least one electrode is a focused tripolar electrode with a center cathodic contact and flanking anodic contacts.

11. The system of claim 1, wherein the time is 30 minutes or less and the at least partial block of the smaller-diameter nerve fibers lasts four hours or less after the application.

12. The system of claim 1, wherein the time is 30 minutes and the DC is applied in five minute burst over the time, wherein the application is repeated up to four or five times a day.

13. The system of claim 1, wherein the time is from 1 minute to 2 minutes, wherein the application is repeated up to 10 times a day.

14. The system of claim 1, wherein the amplitude is less than 1 mA.

15. A method for blocking conduction in small-diameter nerve fibers while leaving large-diameter, myelinated nerve fibers at least partially unblocked and able to conduct, the method comprising:
generating, by a current generator, at least one direct current (DC) with an amplitude less than 15 mA for a time; and
applying, by at least one electrode coupled to the current generator, the DC to a nerve for the time,
wherein application of the DC to the nerve for the time creates a spatially restricted DC field based on the DC, such that the spatially restricted DC field is configured to at least partially block conduction in the small-diameter nerve fibers of the nerve while leaving the large-diameter, myelinated nerve fibers of the nerve able to conduct.

16. The method of claim 15, wherein the conduction is blocked in the small-diameter nerve fibers that cause neuropathic pain while conduction in the large-diameter, myelinated nerve fibers is at least partially able to continue.

17. The method of claim 15, wherein the conduction is at least partially blocked in the small-diameter nerve fibers that cause spasticity while conduction in the large-diameter, myelinated nerve fibers is at least partially able to continue.

18. The method of claim 15, wherein the conduction is at least partially blocked in the small-diameter nerve fibers that cause unwanted autonomic activity while conduction in the large-diameter, myelinated nerve fibers is at least partially able to continue.

19. The method of claim 15, wherein the spatially restricted DC field is a focused field that has a thinner length than a traditional broad DC field.

20. The method of claim 19, wherein the focused field is thinner than a distance between 5 nodes of Ranvier in the large-diameter, myelinated nerve fiber.
